(19) United States
(12) Patent Application Publication
(10) Pub. No.: US 2024/0358639 A1
(43) Pub. Date: Oct. 31, 2024

(54) NANOPARTICLES FOR CANCER TREATMENT

(71) Applicant: Immix Biopharma, Inc., Los Angeles, CA (US)

(72) Inventor: Ilya Rachman, Los Angeles, CA (US)

(21) Appl. No.: 18/577,129

(22) PCT Filed: Jul. 7, 2022
(86) PCT No.: PCT/US2022/036419

§ 371 Date: Jan. 5, 2024

Related U.S. Application Data
(60) Provisional application No. 63/219,348, filed on Jul. 7, 2021.

(51) Int. Cl. A61K 9/127; A61K 9/00; A61K 9/51; A61K 31/12; A61K 31/704; A61K 39/00; A61P 35/00; B82Y 5/00; C07K 16/28

(52) U.S. Cl. CPC A61K 9/1271; A61K 9/0019; A61K 9/5123; A61K 31/12; A61K 31/704; A61P 35/00; C07K 16/2818; C07K 16/2827; A61K 2039/505; B82Y 5/00

(57) ABSTRACT

The invention disclosed herein relates to nanoparticles containing one or more polykinase inhibitors and methods of using the same in combination with checkpoint inhibitors to treat cancer.

This application claims priority to U.S. Provisional Application Ser. No. 63/219,348, filed Jul. 7, 2021, which is hereby incorporated by reference in its entirety.

### TECHNICAL FIELD OF THE INVENTION

The invention disclosed herein generally relates to nanoparticles containing one or more polykinase inhibitors and methods of using the same in combination with checkpoint inhibitors and/or cancer antibodies to treat cancer.

### BACKGROUND TO THE INVENTION

Programmed death receptor 1 (PD-1) is an immunoinhibitory receptor that is primarily expressed on activated T and B cells. Interaction with its ligands has been shown to attenuate T-cell responses both in vitro and in vivo. Blockade of the interaction between PD-1 and one of its ligands, PD-L1, has been shown to enhance tumor-specific CD8− T-cell immunity and may therefore be helpful in clearance of tumor cells by the immune system.

The ligands for PD-1 (PD-L1 and PD-L2) are constitutively expressed or can be induced in a variety of cell types, including non-hematopoietic tissues as well as various tumor types. PD-L1 is expressed on B, T, myeloid and dendritic cells (DCs), but also on peripheral cells, like microvascular endothelial cells and non-lymphoid organs like heart, lung etc. In contrast, PD-L2 is only found on macrophages and DCs. The expression pattern of PD-1 ligands is suggestive of a role for PD-1 in maintaining peripheral tolerance and may serve to regulate self-reactive T- and B-cell responses in the periphery.

Therapeutic blockade of the PD-1 pathway may be helpful in overcoming immune tolerance. Such selective blockade may be of use in the treatment of cancer or infection as well as in boosting immunity during vaccination (either prophylactic or therapeutic). For example, blockade of the PD-1/PD-L1 interaction could lead to enhanced tumor-specific T-cell immunity and therefore be helpful in clearance of tumor cells by the immune system.

Development of immunotherapy modalities over the last several decades, including immune checkpoint inhibitors, such as anti-PD-1 and anti-PD-L1 antibodies, represents a major advancement in cancer treatment. Checkpoint inhibitors have become a mainstay treatment in several solid tumors producing impressive long-term remissions in a significant minority of patients. Their efficacy in patients with solid tumors however remains quite unpredictable, with a majority of patients not deriving benefit from this therapeutic class. It is therefore desirable to extend the clinical benefit of these therapies.

### SUMMARY OF INVENTION

Some embodiments of the invention relate to a nanoparticle that can include a hydrophilic PEG polymer linked to a hydrophobic polymer lipid core and one or more polykinase inhibitors and, optionally, a chemotherapy agent. In some embodiments, the compositions disclosed herein comprise a nanoparticle, such as PEG2000 length, and a curcuminoid or curcuminoid analog, derivative or salt thereof or curcuminoid complex, optionally co-loaded with doxorubicin or a pharmaceutical equivalent, analog, derivative, and/or salt thereof, for administration prior to anti-PD1 antibody. In some embodiments, the nanoparticle is capable of being used in combination with an anti-PD-1/PD-L1/PD-L2 antibody for the treatment of cancer. In some embodiments, the co-administration of the nanoparticle and the anti-PD-1/PD-L1/PD-L1/PD-L2 antibody causes an increase in efficacy of the antibody compared to administration of the antibody without the nanoparticle.

In some embodiments, the nanoparticle has a size from about 10 nm to about 20 nm, or from about 10 nm to about 30 nm, or from about 10 nm to about 40 nm, or from about 10 nm to about 50 nm, or from about 15 nm to about 45 nm, or from about 15 nm to about 35 nm, or from about 15 nm to about 30 nm. In some embodiments, the nanoparticle has a size from about 20 nm to about 60 nm, or from about 20 nm to about 50 nm, or from about 20 nm to about 40 nm, or from about 20 nm to about 30 nm. In some embodiments, the nanoparticle has an average diameter of less than about 60 nm, or less than about 50 nm, or less than about 40 nm. In some embodiments, the nanoparticle has an average diameter of less than about 30 nm, or less than about 20 nm.

In some embodiments, the one or more kinase inhibitors can be a curcuminoid or curcuminoid analog, derivative or salt thereof or combination thereof, or a curcuminoid complex. In some embodiments, the chemotherapy agent can be doxorubicin or a pharmaceutical equivalent, analog, derivative, and/or salt thereof.

Some embodiments of the invention relate to a pharmaceutical composition that includes a micelle construct of a curcuminoid complex co-loaded with doxorubicin. In some embodiments, the micelle construct is between about 10 nm and 20 nm. In some embodiments, the micelle construct is between about 20 nm and 60 nm. In some embodiments, the micelle construct is less than about 30 nm.

In some embodiments, the composition further includes a pharmaceutically acceptable carrier. In some embodiments, the composition includes PEG2000.

Some embodiments of the invention relate to methods of treating cancer in a subject. The methods can include co-administering a therapeutically effective dosage of the nanoparticle in combination with anti-PD-1/PD-L1/PD-L2 antibody to the subject. In some embodiments, the subject is a human.

In some embodiments, the cancer is selected from the group consisting of Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck (Nasopharyngeal) Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CIVIL), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenström Macroglobulinemia, or Wilms Tumor.

In some embodiments, the nanoparticle composition can be administered prior to anti-PD-1/PD-L1/PD-L2 antibody. In some embodiments, the nanoparticle composition can be administered at the same time with an anti-PD-1/PD-L1/PD-L2 antibody. In some embodiments, the nanoparticle composition can be administered prior to and at the same time as an anti-PD-1/PD-L1/PD-L2 antibody.

In some embodiments, the nanoparticle composition can be administered intravenously. In some embodiments, the concentration of curcuminoids administered intravenously to the subject can be at least 5 mg/m². In some embodiments, the concentration of curcuminoids administered intravenously to the subject can be at least 5 mg/m²-150 mg/m² per dose.

In some embodiments, the dosing of the nanoparticle is once a day for 5 days every 28 days. In some embodiments, the dosing of the nanoparticle is once a day for 6 to 14 days.

### BRIEF DESCRIPTION OF THE DRAWINGS

FIG. 1 depicts a dosing schedule and survival data for experiments related to the invention used in a mouse model of pancreatic cancer.

FIG. 2 depicts imaging of the tumors in mice used in experiments related to the invention.

### DETAILS OF INVENTION

While the ability of checkpoint inhibitors to marshal the immune system to attack cancer can produce impressive and long-lasting responses, these drugs face significant challenges on the molecular, tissue and organismic level. Embodiments of the invention disclosed herein relate to compositions and methods for treating cancer by administering a combination of nanoparticles including one or more hydrophobic kinase inhibitors and optionally a chemotherapeutic agent, and optionally co-administering an anti-PD-1 antibody. The advantage of the combination is to overcome the complex layers of multiple negative feedback loops that prevent most patients from responding to anti-PD-1 treatments.

### Nanoparticles

In some embodiments, the nanoparticles can be liposomes that include an aqueous compartment enclosed by at least one lipid bilayer. When lipids that include a hydrophilic headgroup are dispersed in water, they spontaneously form bilayer membranes referred to as lamellae. The lamellae are composed of two monolayer sheets of lipid molecules with their non-polar (hydrophobic) surfaces facing each other and their polar (hydrophilic) surfaces facing the aqueous medium.

In some embodiments, the nanoparticles have a size from about 10 nm to about 400 nm, or from about 25 nm to about 350 nm, or from about 50 nm to about 300 nm, or from about 100 nm, to about 250 nm, or from about 150 nm to about 200 nm, or from about 100 nm to about 400 nm, or from about 10 nm to about 100 nm, or from about 10 nm to about 90 nm, or from about 10 nm to about 80 nm, or from about 10 nm to about 70 nm, or from about 10 nm to about 60 nm, or from about 10 nm to about 50 nm, or from about 10 nm to about 40 nm, or from about 10 nm to about 30 nm, or from about 20 nm to about 60 nm, or from about 20 nm to about 50 nm, or from about 20 nm, to about 40 nm, or from about 25 nm to about 50 nm, or from about 25 nm to about 40 nm, or from about 25 nm to about 35 nm. In some embodiments, the nanoparticles have a size of about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 125 nm, about 150 nm, about 175 nm, about 200 nm, about 225 nm, about 250 nm, about 275 nm, about 300 nm, about 325 nm, about 350 nm, about 375 nm, or 400 nm. In some embodiments, the nanoparticle has an average diameter of less than about 50 nm, or less than about 45 nm, or less than about 40 nm, or less than about 35 nm, or less than about 30 nm, or less than about 25 nm, or less than about 20 nm.

In some embodiments, the nanoparticles are micelle constructs that include amphiphilic polymers with a hydrophilic exterior and a hydrophobic interior compartment. When these amphiphilic polymers are exposed to an aqueous environment, they spontaneously assemble into single layer complexes with their non-polar hydrophobic portions facing the interior core of the nanoparticle. In some embodiments, the nanoparticle has a size from about 10 nm to about 60 nm. In some embodiments, the nanoparticle has a size from about 10 nm to about 20 nm, or from about 10 nm to about 30 nm, or from about 10 nm to about 40 nm, or from about 10 nm to about 50 nm, or from about 15 nm to about 45 nm, or from about 15 nm to about 35 nm, or from about 15 nm to about 30 nm. In some embodiments, the nanoparticle has a size from about 20 nm to about 60 nm, or from about 20 nm to about 50 nm, or from about 20 nm to about 40 nm, or from about 20 nm to about 30 nm. In some embodiments, the nanoparticle has an average diameter of less than about 60 nm, or less than about 50 nm, or less than about 40 nm. In some embodiments, the nanoparticle has an average diameter of less than about 30 nm, or less than about 20 nm.

### Lipids

In some embodiments, the nanoparticles provided herein comprise a lipid. Suitable lipids include fats, waxes, steroids, cholesterol, fat-soluble vitamins, monoglycerides, diglycerides, phospholipids, sphingolipids, glycolipids, cationic or anionic lipids, derivatized lipids, and the like.

Suitable phospholipids include, but are not limited to, phosphatidylcholine (PC), phosphatidic acid (PA), phosphatidylethanolamine (PE), phosphatidylglycerol (PG), phosphatidylserine (PS), and phosphatidylinositol (PI), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(Nmaleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin.

In some embodiments, the lipids are derivatized lipids, such as PEGylated lipids. Derivatized lipids can include, for example, DSPE-PEG2000, cholesterol-PEG2000, DSPE-polyglycerol, or other derivatives generally known in the art. In some embodiments, the nanoparticles disclosed herein comprise a PEG polymer, such as PEG2000. The length of the polymer determines the micelle size, which promotes its tissue penetration and improves delivery.

### Kinase Inhibitors

In some embodiments, the nanoparticles disclosed herein include one or more hydrophobic kinase inhibitors. The hydrophobic kinase inhibitor can be a curcuminoid or curcuminoid analog, derivative or salt thereof. The curcuminoid(s) can be natural or synthetic. The kinase inhibitor can be a synthetic analog of curcumin.

Curcuminoids are polyphenolic pigments and include curcumin, demethoxycurcumin, and bisdemethoxycurcumin.

As used herein curcumin is also known as diferuloylmethane or (E,E)-1,7-bis (4 hydroxy-3-methoxyphenyl)-1,6-heptadiene-3,5,-dione. Curcumin can be derived from a natural source, the perennial herb Curcuma longa L., which is a member of the Zingiberaceae family.

Curcumin is soluble in ethanol, alkalis, ketones, acetic acid and chloroform. It is insoluble in water. Curcumin is therefore lipophilic, and generally readily associates with lipids, e.g., many of those used in the colloidal drug-delivery systems of embodiments of the invention disclosed herein. In some embodiments, curcumin can also be formulated as a metal chelate.

As used herein, curcumin analogues are those compounds which due to their structural similarity to curcumin, exhibit effects similar to that of curcumin. Curcumin analogues include, but are not limited, to Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis (2-hydroxy naphthyl)-1,6-heptadiene-2,5-dione (2-hydroxyl naphthyl curcumin), 1,1-bis (phenyl)-1,3,8,10 undecatetraene-5,7-dione (cinnamyl curcumin), and the like. Curcumin analogues also include isomers of curcumin, such as the (Z,E) and (Z,Z) isomers of curcumin. In some embodiments, curcumin metabolites are used. Known curcumin metabolites include, but are not limited to, glucoronides of tetrahydrocurcumin and hexahydrocurcumin, and dihydroferulic acid. In some embodiments, curcumin analogues or metabolites can be formulated as metal chelates, especially copper chelates. Other derivatives of curcumin, curcumin analogues and curcumin metabolites appropriate for use in embodiments of the invention disclosed herein will be apparent to one of skill in the art based on the instant disclosure.

In some embodiments, the nanoparticles disclosed herein include a combination of different kinase inhibitors. In some embodiments, the inhibitor can be EF24, EF31 and other compounds disclosed in U.S. Pat. No. 7,842,705, which is hereby incorporated by reference in its entirety.

### Chemotherapeutic Agents

In some embodiments, the nanoparticles disclosed herein can optionally further include one or more chemotherapy agents. Exemplary chemotherapeutic agents include, but are not limited to, an anthracycline (e.g., doxorubicin). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide), and the like.

In some embodiments, the nanoparticles disclosed herein are co-loaded with one or more polykinase inhibitors and one or more chemotherapeutic agents. For example, in some embodiments, the nanoparticle is co-loaded with one or more polykinase inhibitors and doxorubicin. In some embodiments, the nanoparticle is a micelle construct of curcuminoid complex co-loaded with doxorubicin (“IMX-110”).

### Therapeutic Compositions

In some embodiments, therapeutic compositions including the nanoparticle described herein are provided. The compositions can include a pharmaceutically acceptable carrier and, optionally, other desired components. The carrier(s) disclosed herein are acceptable in the sense of being compatible with the other ingredients of the formula and not deleterious to the recipient thereof. Selection of appropriate carriers, e.g., phosphate buffered saline and the like, are well within the skill of those in the art. In some embodiments, the carrier can include PEG2000.

Similarly, one skilled in the art can readily select appropriate stabilizers, preservatives, and the like for inclusion in the composition. Any route of administration known in the art can be employed for the administration of the nanoparticle, e.g., subcutaneous, intraperitoneal, intravenous (i.v.), intramuscular (i.m.), intrasternal, intratumoral, infusion, oral, intramuscular, intranasal, and the like. In some embodiments, the composition is suitable for delivery by i.v. administration.

### Method of Making the Nanoparticle

In some embodiments, methods of producing the nanoparticles described herein are provided. In some embodiments, the method can include mixing the phospholipid, polyphenolic kinase inhibitors (curcuminoids) or other hydrophobic kinase inhibitors with an organic solvent to solubilize the mixture. If a chemotherapeutic agent is used, the agent is included in the mixture. After that, the solvent can be evaporated by known methods and the mixture rehydrated in PBS. The physicochemical properties, such as particle size, surface charge, the encapsulation efficiency and content can be determined according to known methods. Further information can be found in Sarisozen et. al., European Journal of Pharmaceutics and Biopharmaceutics 108 (2016) 54-67, which is hereby incorporated by reference in its entirety.

### Method of Using the Nanoparticle

In some embodiments, methods of using the nanoparticle compositions disclosed herein are provided herein. In some embodiments, the methods relate to treating cancer in a subject by administering an effective amount of the nanoparticle compositions disclosed herein.

The term “effective amount” or “therapeutically effective amount” are used interchangeably herein, and refer to an amount of a compound, formulation, material, or composition, as described herein effective to achieve a particular biological result.

The term “cancer” refers to a disease characterized by the rapid and uncontrolled growth of aberrant cells. Cancer cells can spread locally or through the bloodstream and lymphatic system to other parts of the body. Examples of cancers treated according to the methods described herein include, but are not limited to, Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck (Nasopharyngeal) Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CIVIL), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenström Macroglobulinemia, Wilms Tumor, and the like.

The term “cancer” refers to all types of cancerous growths or oncogenic processes, metastatic tissues or malignantly transformed cells, tissues, or organs, irrespective of histopathologic type or stage of invasiveness.

In some embodiments, the cancer treated according to the methods disclosed herein is characterized by expression of a PD-1 ligand, e.g., PD-L1 or PD-L2, on a cancer cell or in a tumor microenvironment.

In some embodiments, the methods disclosed herein include co-administration of a composition including the nanoparticles provided herein and an anti-PD-1/PD-L1/PD-L2 antibody. As used herein “anti-PD1/PD-L1/PD-L1” refers to any antibody or antibody fragment that blocks binding of PD-L1 and/or PD-L2 to PD-1.

The terms “co-administration” or “combined administration” or the like as utilized herein are meant to encompass administration of the selected therapeutic agents to a single patient and are intended to include treatment regimens in which the agents are not necessarily administered by the same route of administration or at the same time. In some embodiments of the methods described herein, the nanoparticle composition and anti-PD-1/PD-L1/PD-L2 can be co-administered.

In some embodiments, co-administration of the nanoparticles disclosed herein and an anti-PD-1/PD-L1/PD-L2 antibody has synergistic effects.

The terms “synergistic” and “synergistically effective” are used herein to mean a biological effect created from the application of two or more agents that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15 (1): 20-23, 1967, the entire contents of which is fully incorporated by reference herein.

In some embodiments, the synergistic effect can be the enhanced anti-tumor response of the PD-1/PD-L1/PD-L2 antibody by the compositions disclosed herein due to the combination of an inhibitor of NF-kB and Stat3 activation, curcumin, and an apoptosis inducer/topoisomerase inhibitor, Doxorubicin. In some embodiments, the synergistic effect are demonstrated by the use of a lower PD-1/PD-L1/PD-L2 antibody dose compared to standard doses. For example, in some embodiments, the antibody dose can be less than the standard dose but still be as effective or even more effective when used in combination with the compositions disclosed herein. For example, the antibody dose can be about 50%, or about 55%, or about 60%, or about 65%, or about 70%, or about 75%, or about 80%, or about 85%, or about 90%, or about 95% of the standard dose.

In some embodiments, the compositions disclosed herein comprise a nanoparticle, such as PEG2000 length, and a curcuminoid or curcuminoid analog, derivative or salt thereof or curcuminoid complex, optionally co-loaded with doxorubicin or a pharmaceutical equivalent, analog, derivative, and/or salt thereof, for administration prior to anti-PD1 antibody. Thousands of combinations have been tried by others in vitro, but none have shown the ability to functionalize this combination approach in vivo, let alone in human patients, as has been demonstrated by the inventors of the instant invention. Further, the dose of doxorubicin used herein is significantly lower than the commonly used doxorubicin dose, while the efficacy is better, pointing to the unanticipated synergy not previously disclosed in any prior art.

In some embodiments, the nanoparticle compositions disclosed herein are administered prior to administration of the anti-PD-1/PD-L1/PD-L2 antibody. In some embodiments, the nanoparticle compositions are administered at the same time as administration of the anti-PD-1/PD-L1/PD-L2 antibody. In some embodiments, the nanoparticle compositions are administered prior to and at the same time as administration of the anti-PD-1/PD-L1/PD-L2 antibody.

In some embodiments, co-administration of the nanoparticle compositions disclosed herein and anti-PD-1/PD-L1/PD-L2 antibodies increases the efficacy of the antibody compared to administration of the antibody without the nanoparticle composition.

In some embodiments, the methods disclosed herein include administration of a composition comprising a nanoparticle that includes one or more hydrophobic kinase inhibitors to a subject. In some embodiments, administration can be intravenous, oral, inhaled, intranasal, rectal, topical, and the like.

In some embodiments, the kinase inhibitor is a curcuminoid administered in a dose of from about 0.01 mg/kg of an individual's body weight to about 500 mg/kg of an individual's body weight. For example, the curcuminoid can be administered in a dose of from about 0.01 mg/kg, about 1 mg/kg, about 10 mg/kg, about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, or about 500 mg/kg. In some embodiments, the kinase inhibitor is a curcuminoid administered in a dose of from about 5 mg/m² to about 150 mg/m² per dose. For example, the curcuminoid can be administered in a dose of from about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 110 mg/m², about 120 mg/m², about 130 mg/m², about 140 mg/m², or about 150 mg/m² per dose.

The curcumin can be selected from Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis (4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin), 1,1-bis(phenyl)-1,3,8,10 undecatetraene-5,7-dione, and the like.

In some embodiments, the dosing regimen of the nanoparticle composition disclosed herein is, for example, once a day, twice a day, three times per day, or every 2, 3, 4, 5, 6, 7, or 8 hours. In some embodiments the treatment length can be 5 days, 7 days, 10 days, two weeks, three weeks, four weeks, or from about 5 days to 28 days.

In some embodiments, co-treatment with anti-PD-1/PD-L1/PD-L2 antibody can occur after or at the same time as the start of administration of the nanoparticle composition disclosed herein. For example, the anti-PD-1/PD-L1/PD-L2 antibody can be administered on day 0, 1, 2, 3, 4, 5, 6, 7, 8, 9, 10 or more after the nanoparticle composition disclosed herein is administered. Administration of anti-PD-1/PD-L1/PD-L2 antibody can be standard to or less than what is currently used in the art (see Tawbi, Hussein A et al. “Pembrolizumab in advanced soft-tissue sarcoma and bone sarcoma (SARC028): a multicentre, two-cohort, single-arm, open-label, phase 2 trial.” The Lancet. Oncology vol. 18.11 (2017): 1493-1501. doi:10.1016/S1470-2045 (17) 30624-1 and Kang, Yoon-Koo et al. “Nivolumab in patients with advanced gastric or gastro-oesophageal junction cancer refractory to, or intolerant of, at least two previous chemotherapy regimens (ONO-4538-12, ATTRACTION-2): a randomized, double-blind, placebo-controlled, phase 3 trial.” Lancet (London, England) vol. 390,10111 (2017): 2461-2471. doi:10.1016/S0140-6736 (17) 31827-5; the entirety of each of which is incorporated by reference herein. In some embodiments, the co-administration regimen of the nanoparticle composition disclosed herein and the anti-PD-1/PD-L1/PD-L2 antibody can be repeated. For example, the regimen can be repeated every 1, 2, 3, 4, 5, 6 weeks for 2, 3, 4, 5, 6, 7, 8, 9, 10, 11, 12 months or more.

### Programmed Death Receptor 1 (PD-1)

In some embodiments, the invention provides isolated antibodies and antibody fragments that bind to PD-1. In some embodiments, the antibody or antibody fragments block binding of PD-L1 and PD-L2 to PD-1.

### Advantages

The inventors of the invention disclosed herein have demonstrated the feasibility of using nanoparticles with polykinase inhibitors and chemotherapy agents, exemplified by the use of a micelle co-loaded with curcuminoid complex and doxorubicin (IMX-110) as a pretreatment for, or co-treatment with, anti-PD-1 antibodies to improve the anticancer efficacy of these drugs in hard-to-treat advanced solid tumors. The use of the immuno-intact KPC pancreatic cancer model in the Examples below highlights the potency of IMX-110 in overcoming the immunosuppressive effects of advanced cancer countering the anti-PD-1 effects. The novel combination treatment disclosed in the present application represents a promising new direction in the treatment of advanced solid cancers.

A counterintuitive aspect of the invention disclosed herein is to combine seemingly mutually inhibitory agents such as curcuminoids, which inhibit NF-kb and Stat-3 activation, and anti-PD1 antibodies, which activate immune response through NF-kb and Stat3 activation in immune cells. The prevailing thinking prior to the instant invention was that any anti-NF-kB and anti-Stat3 drugs would impair anti-tumor immune response, not stimulate it, as has been demonstrated by the instant inventors.

One of ordinary skill in the art will appreciate the critical importance of NF-kb and Stat3 transcriptional activation. It would be highly counterproductive to administer the anti-NF-kB and anti-Stat3 agents without a precise spatial, temporal and cell-cycle specific manner and expect to observe a meaningful anti-cancer immune response. It is, therefore, highly surprising that by combining IMX-110 (which combines not only an inhibitor of NF-kB and Stat3 activation (Cur) but also an apoptosis inducer/topoisomerase inhibitor Dox) with a PD-1 antibody, the inventors observed an enhanced anti-tumor response, even at a lower dose of PD-1 antibody than those in published reports as described in Example 1.

Anti-cancer immune response promoted by anti-PD1 antibodies is opposed by many processes active in cancer. One such opposing factor is the upregulation of exosome production in cancer patients. These exosomes express PD-1/PD-L1 on their surface and hence act as adsorbers of the administered anti-PD-1 antibodies, hindering their intended effects (see Yin, Zi et al., “Mechanisms underlying low-clinical responses to PD-1/PD-L1 blocking antibodies in immunotherapy of cancer: a key role of exosomal PD-L1.” Journal for immunotherapy of cancer vol. 9.1 (2021): e001698. doi:10.1136/jitc-2020-001698; the entirety of which is incorporated by reference herein). Also limiting the effector functions of the immune system in the fight against cancer is the acidic environment present in tumor tissues (see Huber, Veronica et al. “Cancer acidity: An ultimate frontier of tumor immune escape and a novel target of immunomodulation.” Seminars in cancer biology vol. 43 (2017): 74-89. doi:10.1016/j.semcancer.2017.03.001; the entirety of which is incorporated by reference herein). Both, exosomal production and acidification are, at least partially, activated by NF-kB and Stat3. Thus, an advantage of the invention disclosed herein is the appropriately timed and dosed administration of an NF-kB/Stat3 inhibitor relative to an anti-PD1 antibody to maximize their desired effects.

### EXAMPLES

### Example 1

To demonstrate the proof of concept that anticancer efficacy of anti-PD-1 antibody can be improved by pretreatment with a drug that can block the cancer-protective immune mechanisms that impair anti-PD-1 efficacy, the inventors combined micelles co-loaded with curcuminoid complex and doxorubicin (IMX-110) with a murine anti-PD-1 antibody in a KPC mouse genetic immunointact model of pancreatic cancer (See Lee et al. Curr Protoc Pharmacol. 2016; 73:14.39.1-14.39.20, which is hereby incorporated by reference in its entirety). This mouse model behaves similarly to human pancreatic cancer, where it is only minimally responsive to chemotherapy, radiation, or immuno-oncology drugs, including anti-PD-1 antibodies. Being immuno-intact, these mice represent an ideal model to study the effects of immunological drugs.

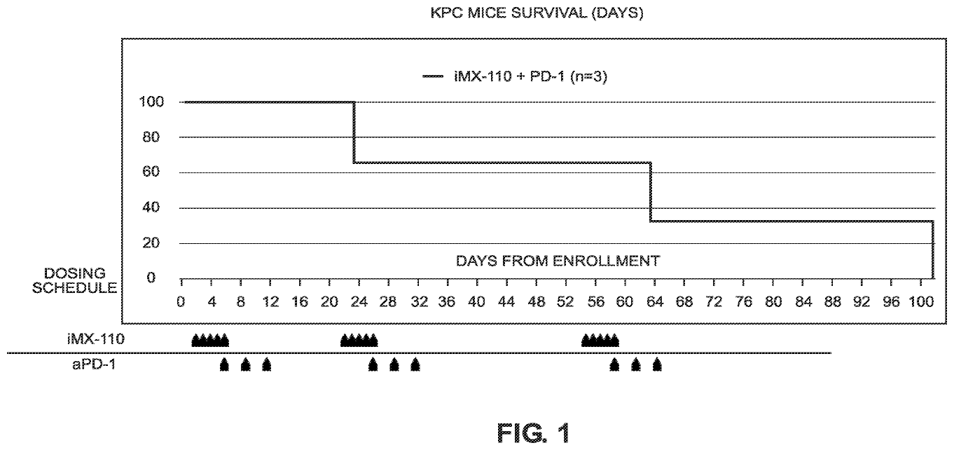

Mice with a mutant KRAS under the control of a pancreas-specific promoter were used for this study. Once mice developed visible lesions with in vivo luciferase assays representing pancreatic cancer, they were treated with a combination of IMX-110 and anti-PD-1 antibody. The treatment schedule was five (5) daily injections of IMX-110 at 1.5 mg/kg of Doxorubicin/6 mg/kg of Curcumin per dose) days 1-5 and 100 microgram of murine anti-PD1 antibody i.v. given on Days 5, 8 and 11. These cycles were repeated every three (3) weeks and the survival duration as well as the clinical status of mice was accessed by observation (see FIG. 1).

The IMX-110+anti-PD-1 antibody combination treatment showed prominent reduction in tumor masses using just 50% of the commonly administered murine anti-PD1 antibody published in the literature (Winograd, Rafael et al., “Induction of T-cell Immunity Overcomes Complete Resistance to PD-1 and CTLA-4 Blockade and Improves Survival in Pancreatic Carcinoma.” Cancer immunology research vol. 3.4 (2015): 399-411. doi:10.1158/2326-6066.CIR-14-0215; http://cancerimmunolres.aacrjournals.org/content/3/4/399.long), which is hereby incorporated by reference in its entirety.

In two out of three mice, there was significant reduction in the high intensity luciferase signal indicating reduction in pancreatic mass size. In addition, the clinical status of all mice at this 40-week stage appeared better than typically observed in these mice.

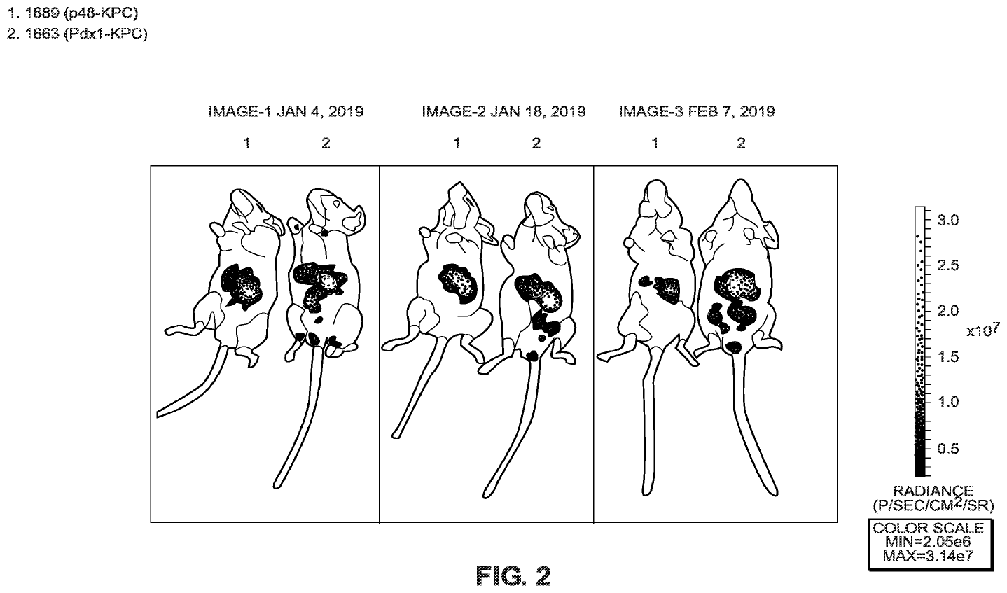

FIG. 2 shows imaging data post 2nd cycle of IMX-110 and anti-PD1 antibody. In one mouse, the tumor size continued to shrink and the other remained clinically doing well while displaying seeming tumor enlargement on the scan image.

### Example 2

Experiments are conducted to demonstrate synergy and determine optimal dosing levels of micelles loaded with curcuminoid complex (with or without doxorubicin) and optimal timing of its administration relative to administration of the anti-PD1 antibody. To maximally explore the synergy between a nanoparticle encapsulating polykinase inhibitors (with or without doxorubicin) and anti-PD1 antibody, a myriad of factors that influence the clinical outcome of their interactions as discussed above must be considered.

An immuno-intact cancer models is used. Mice are intravenously dosed with IMX-110 on Days 1-5, then nanoparticles with polykinase inhibitor alone (without Dox) for 7-14 days. Dose ranges tested in mice include Dox: 0.5 mg/kg-3 mg/kg; Curcuminoids: 2 mg/kg-100 mg/kg at 50% and 100% of their standard published doses. Anti-PD1 antibody is administered weekly. Synergy is observed and optimal dosing schedules are determined.

### Example 3

Human patients with advanced solid tumors are treated intravenously with micelles co-loaded with curcuminoid complex and doxorubicin and anti-PD1 antibody. Doxorubicin dose range is: 2.5 mg/m²-10 mg/m², with curcumin dose ranging between 10 mg/m² and 150 or more mg/m² per dose. The dosing schedule is once a day for five (5) days every 28 days. Anti-PD1 antibody is administered every 14 to 21 days in human patients at 50% and 100% of their standard known doses. Therapeutic synergy is observed and optimal dosing schedules are confirmed.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A nanoparticle comprising a hydrophilic PEG polymer linked to a hydrophobic polymer lipid core and one or more polykinase inhibitors and, optionally, a chemotherapy agent.

2. The nanoparticle of claim 1, wherein the hydrophilic PEG polymer is PEG2000.

3. The nanoparticle of claim 1, wherein the nanoparticle has a size of about 20 nm to about 50 nm.

4. The nanoparticle of claim 1, wherein the one or more kinase inhibitors is selected from a curcuminoid or curcuminoid analog, derivative or salt thereof or combination thereof.

5. The composition of claim 1, wherein the chemotherapy agent is doxorubicin or a pharmaceutical equivalent, analog, derivative, and/or salt thereof.

6. A pharmaceutical composition comprising a micelle construct of a curcuminoid complex co-loaded with doxorubicin.

7. The composition of claim 6, wherein the micelle construct is between 10 nm and 20 nm.

8. The composition of claim 6, wherein the micelle construct is between 20 nm and 60 nm.

9. The composition of claim 6, wherein the micelle construct is less than 30 nm.

10. The composition of claim 6, further comprising a pharmaceutically acceptable carrier comprising PEG2000.

11. A method of treating cancer in a subject, comprising co-administering a therapeutically effective dosage of the nanoparticle of claim 1 in combination with anti-PD-1/PD-L1/PD-L2 antibody to the subject.

12. The method of claim 11, wherein the subject is a human.

13. The method of claim 11, wherein the cancer type is selected from the group consisting of Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck (Nasopharyngeal) Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Myelodysplastic Syndrome, Myelodysplastic/Myeloproliferative Neoplasm, Chronic Myelogenous Leukemia (CIVIL), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, and Wilms Tumor.

14. The method of claim 11, wherein the nanoparticle of claim 1 is administered prior to anti-PD-1/PD-L1/PD-L2 antibody.

15. The method claim 11, wherein the nanoparticle of claim 1 is administered at the same time with an anti-PD-1/PD-L1/PD-L2 antibody.

16. The method of claim 11, wherein the nanoparticle of claim 1 is administered intravenously.

17. The method of claim 11, wherein the concentration of curcuminoid administered intravenously to the subject is at least 5 mg/m² per dose.

18. The method of claim 11, wherein the concentration of curcuminoid administered intravenously to the subject is 5 mg/m² to 150 mg/m² per dose.

19. The method of claim 11, wherein the nanoparticle dosing schedule is once a day for 5 days every 28 days

20. The method of claim 11, wherein the nanoparticle dosing schedule is once a day for 6 to 14 days every 28 days
